(19) United States
(12) Patent Application Publication
(10) Pub. No.: US 2023/0240665 A1
(43) Pub. Date: Aug. 3, 2023

(54) MAGNETICALLY ACTUATED CAPSULE

(71) Applicants: The Governing Council of the University of Toronto, Toronto (CA); The Hospital for Sick Children, Toronto (CA)

(72) Inventors: Eric Diller, York (CA); Peyman Shokrollahi, North York (CA); John Parkinson, Toronto (CA)

(21) Appl. No.: 18/003,256

(22) PCT Filed: Jun. 28, 2021
(86) PCT No.: PCT/CA2021/050887

§ 371 Date: Dec. 23, 2022

Related U.S. Application Data
(60) Provisional application No. 63/045,794, filed on Jun. 29, 2020.

(51) Int. Cl. A61B 10/00; B29C 39/00; B29C 39/02; A61B 5/00

(52) U.S. Cl. CPC A61B 10/0045; B29C 39/003; B29C 39/021; A61B 5/6861; B29K 2083/005

(57) ABSTRACT

A magnetically actuated capsule comprising two portions pivotally connected to each other for allowing tetherless reconfiguration of the capsule between a closed configuration and an open configuration inside a body cavity by an external magnet. The capsule can be used for noninvasive sampling of microbiomes and liquid within a body cavity, or to release a cargo within the body cavity. Methods of using and manufacturing the capsule are disclosed.

### FIELD OF THE INVENTION

The present disclosure relates generally to magnetically actuated capsules. The present disclosure relates more particularly to a magnetically actuated capsule for noninvasive sample retrieval and/or dispensing in the body cavity, for example the gastrointestinal (GI) tract, and methods for operating the same.

### BACKGROUND OF THE INVENTION

The mammalian gastro-intestinal tract (GI) is home to a complex community of microbes, termed the microbiome. Increasingly, the composition and function of the GI microbiome in humans has been linked to a large range of diseases including diabetes and inflammatory bowel disease (IBD) [1-5]. From a livestock perspective, global bans on the use of dietary supplementation of antibiotics is driving interest in the identification of alternative strategies, such as probiotic feed additives, for promoting growth through the manipulation of the gut microbiome. The key to developing new therapeutic strategies in the case of human disease, or novel feed additives in the case of livestock production, is an improved understanding of the interactions between the gut microbiome and the host immune system. A major challenge to such an understanding is the retrieval of physical samples that inform on the composition and function of the microbiome from physiologically-relevant sites within the GI tract. While the composition and activity of microbes vary dramatically across the GI tract [6], [7], studies of the gut microbiome typically rely on the use of stool samples that are not reflective of intestinal sites relevant to disease or nutrient absorption.

One approach to obtain more informative samples is the use of endoscopy. However, endoscopy is highly invasive, expensive, and in humans typically used only for initial diagnosis. In addition, distal regions of the small intestine are not accessible without the use of invasive instrumentation and procedures. Consequently, there has been much interest in developing less invasive devices capable of sampling from internal regions on a more regular basis [8]-[11]. For example, Kerkhof proposed a device to sample liquids [12], but because it uses pH-sensitive material to control sampling timing, the device may operationally fail due to the dependency on the pH level. Jones et al. [13] recently proposed other ingestible designs for GI tract microbiome sampling, but that actuator system is complex, with a multistage valve system, which increases the risk of failure and limits the potential for scaling to smaller size of capsule. Cui et al. [14] developed a micromachined capsule which has a relatively large size compared to the confined GI tract space, and thus has a risk of retention in the body, similar to endoscopic capsules [15], [16].

Recent advancement of mobile microrobotics suggests new solutions for tasks in healthcare and micro-factory. One major challenge in microrobotics is the control and actuation of microrobots, which is not trivial considering that most microrobots do not have space for onboard power or electronic systems.

Several groups have developed active robotic capsule endoscopes [19], focusing on the development of magnetically actuated controllable endoscopes [20]-[23] and enhanced capsules for drug delivery [24]-[26]. These capsules, however, have limited application and are not adaptable for use for microbiome or digesta sampling. In particular, these capsules are unable to seal tightly, cannot be actuated between an open and closed configuration, or even close altogether once opened, or lack a sampling chamber. A need therefore exists for a stand-alone, small-scale, completely soft microrobot that allows for microbiome sampling across all locations in the GI tract.

In particular, what is needed is a device for noninvasive sampling and/or dispensing in the GI tract that permits (1) remote control of the device in typically inaccessible regions such as the small intestine; and (2) does not require onboard control and power circuits, to allow for downscaling of the size of the device while maximizing the allocation of space for sample collection volume.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a magnetically actuated capsule for noninvasive sample retrieval and/or dispensing in the gastrointestinal (GI) tract. In accordance with one aspect of the present disclosure, there is provided a magnetically actuated capsule comprising two portions pivotally connected to each other for allowing reconfiguration of the capsule between a closed configuration and an open configuration, the portions defining a chamber enclosed within the capsule when the capsule is in the closed configuration, and defining an aperture exposing the chamber to the exterior of the capsule when the capsule is in the open configuration, each portion comprising at least one permanent magnet having a magnetic moment disposed in a direction opposite to the magnetic moment of the at least one permanent magnet in the other portion such that: (i) an inter-magnet attraction force is generated to bias the capsule to the closed configuration, and (ii) the capsule in the closed configuration has a net magnetic moment that aligns with an externally applied magnetic field to actuate reconfiguration of the capsule to the open configuration when said magnetic field is applied.

In accordance with another embodiment of the capsule of the present disclosure, the capsule further comprises a seal for sealing the chamber when the capsule is in the closed configuration, the seal being disposed between the portions in the capsule in the closed configuration.

In accordance with another embodiment of the capsule of the present disclosure, the capsule in the closed configuration comprises a cylindrical midsection, wherein the portions are pivotally connected at a pivot point on the cylindrical midsection of the capsule, and wherein in the closed configuration the magnetic moment of the at least one permanent magnet in each portion is about parallel to a line which intersects the pivot point and which about perpendicularly intersects the cylinder axis of the cylindrical midsection of the capsule.

In accordance with another embodiment of the capsule of the present disclosure, the at least one permanent magnet in each portion is disposed within the portion at an angle αₒ relative to the line which intersects the pivot point, such that the net magnetic moment of the capsule self-aligns with the externally applied magnetic field when said magnetic field is applied.

In accordance with another embodiment of the capsule of the present disclosure, the at least one permanent magnet in each portion is disposed within the portion at an angle αₒ that is between about 5° and about 30° relative to the line which intersects the pivot point.

In accordance with another embodiment of the capsule of the present disclosure, the portions are pivotally connected by a hinge.

In accordance with another embodiment of the capsule of the present disclosure, the capsule comprises a soft outer shell.

In accordance with another embodiment of the capsule of the present disclosure, the hinge and the soft outer shell of the capsule comprise a reinforced composite.

In accordance with another embodiment of the capsule of the present disclosure, the capsule is configured to be swallowed and passed through the intestinal tract of a subject.

In accordance with another embodiment of the capsule of the present disclosure, the capsule is cylindrical in shape comprising an outer diameter of between about 6 mm to 10 mm and a length of between about 9 mm and 13 mm.

In accordance with another embodiment of the capsule of the present disclosure, the capsule comprises an outer diameter of about 8 mm and a length of about 11 mm.

In accordance with another embodiment of the capsule of the present disclosure, the capsule is adapted for collecting a sample from the intestinal tract.

In accordance with another embodiment of the capsule of the present disclosure, the capsule is adapted for collecting a sample from the intestinal tract and the chamber of the capsule further comprises a hydrophilic coating for capturing the sample.

In accordance with another embodiment of the capsule of the present disclosure, the capsule is adapted for delivering one or more agents to the intestinal tract of a subject.

In accordance with another aspect of the disclosure, there is provided a system comprising the capsule according to an embodiment of the present disclosure and an external magnet for generating a magnetic field, wherein application of the magnetic field remotely actuates alignment of the capsule with the externally applied magnetic field to activate reconfiguration of the capsule from the closed configuration to the open configuration.

In accordance with another aspect of the disclosure, there is provided a method of collecting a sample from the intestinal tract of a subject, the method comprising: (a) providing a capsule in the closed configuration according to an embodiment of this disclosure for ingestion by the subject; (b) applying an external magnetic field to the subject to actuate reconfiguration of the capsule to the open configuration, thereby exposing the chamber of the capsule to the exterior of the capsule for collection of the sample in the chamber; (c) removing the external magnetic field to actuate reconfiguration of the capsule to the closed configuration, thereby enclosing the sample in the capsule; and (d) allowing the capsule to transit through the intestinal tract for recovery by stool passage.

In accordance with another aspect of the disclosure, there is provided a method for diagnosing a disease or condition in a subject, the disease or condition being diagnosable by analysis of a liquid digesta sample from the intestinal tract of the subject, the method comprising: (a) providing a capsule in the closed configuration according to an embodiment of the present disclosure for ingestion by the subject; (b) applying an external magnetic field to the subject to actuate reconfiguration of the capsule to the open configuration to thereby expose the chamber of the capsule to the exterior of the capsule and allow a sample from the body cavity to enter the chamber; (c) removing the external magnetic field to actuate reconfiguration of the capsule to the closed configuration, thereby enclosing the sample in the capsule; (d) allowing the capsule to transit through the intestinal tract for recovery by stool passage; and (e) analyzing the sample contained in the recovered capsule so as to diagnose the disease or condition in the subject.

In accordance with another aspect of the disclosure, there is provided a method for delivering an agent to a subject, the method comprising: (a) providing a capsule in the closed configuration according an embodiment of the present disclosure, the chamber of the capsule containing the agent; (b) administering the capsule to the subject by ingestion; and (c) applying an external magnetic field to the subject to actuate reconfiguration of the capsule to the open configuration to dispense the agent contained in the chamber into the intestinal tract of the subject.

In accordance with another aspect of the disclosure, there is provided a method for treating or diagnosing a disease or condition in a subject, the disease or condition being treatable or diagnosable by delivery of a therapeutic or diagnostic agent to the intestinal tract of the subject, the method comprising: (a) providing a capsule in the closed configuration according to an embodiment of the present disclosure, the chamber of the capsule containing the agent; (b) administering the capsule to the subject by ingestion; (c) allowing the capsule to transit through the subject's intestinal tract; and (d) applying an external magnetic field to the subject to actuate reconfiguration of the capsule to the open configuration to dispense the agent contained in the chamber into the intestinal tract of the subject.

In accordance with one embodiment of the method for treating or diagnosing a disease or condition of the present disclosure, the agent is a biological agent or a non-biological agent.

In accordance with another embodiment of the method for treating or diagnosing a disease or condition of the present disclosure, the biological agent is one or more of an antibiotic, fecal matter, nutritional agents, polyclonal/monoclonal antibodies or fragments thereof, recombinant proteins, enzymes RNAi, aptamers, and dendrimers, RNA, and DNA, cytokines, tissue growth factors, gene transfer products, glycosaminoglycans, cells (including stem cells), genetically modified cells (including genetically modified stem cells), bacteria, genetically modified bacteria, probiotics, live biotherapeutic products (LBP), viruses, genetically modified viruses, or nutritional agents.

In accordance with another embodiment of the method for treating or diagnosing a disease or condition of the present disclosure, the non-biologic agent is one or more of a mineral, a vitamin or a small organic molecule.

In accordance with another aspect of the disclosure, there is provided a method of manufacturing a magnetically actuated capsule according to an embodiment of this disclosure, the method comprising: (a) for each portion, providing a mold for forming the portion, the mold comprising a chamber ridge for forming a capsule chamber; (b) mounting at least one permanent magnet to the mold, the at least one permanent magnet being positioned in the mold such that the two portions are biased to magnetically connect to each other to form the capsule; (c) pouring a polymer in a liquid state into the mold and allowing the polymer to solidify, thereby forming the portion comprising the at least one permanent magnet; and (d) contacting the two portions to each other so as to allow the portions to magnetically connect to each other to form the capsule.

In accordance with another embodiment of the method of manufacturing a capsule, the mold further comprises a magnet holder for mounting the at least one permanent magnet, and wherein in step (b) the at least one permanent magnet is mounted on a magnet holder.

In another embodiment of the method of manufacturing a capsule, the method further comprises pivotally joining the portions to each other with a hinge at a pivot point.

In another embodiment of the method of manufacturing a capsule, the step of pivotally joining the portions to each other with a hinge at a pivot point comprises pouring into a hinge mold a composite in a liquid state, the composite being a reinforced form of the polymer used to create the portions of the capsule and being flexible in the solid state, placing the capsule in the hinge mold containing the composite such that a longitudinal strip of the capsule surface spanning both portions contacts the composite, and allowing the composite to solidify, thereby pivotally connecting the portions of the capsule to each other with a flexible composite hinge at a pivot point.

### BRIEF DESCRIPTION OF THE DRAWINGS

These and other features of the invention will become more apparent in the following detailed description in which reference is made to the appended drawings.

FIG. 1A is a perspective view of a magnetically actuated capsule in one embodiment in the closed configuration.

FIG. 1B is a perspective view of the magnetically actuated capsule shown in FIG. 1A in the open configuration.

FIG. 2 is a schematic of the opening operation mechanism of the magnetically actuated capsule shown in FIG. 1 in which the permanent magnets are shown at a slanted angle α₀ and the capsule has an opening angle α₁, where an applied field Bₑₓₜ creates opposing magnetic torques Tₑₓₜ on each of the opposing magnets. This torque balances with the hinge torque Tₕ and the interaction force and torque between the two magnets F₁ and T₁.

FIG. 3 presents a graph depicting the characterization of the B field generated by three external magnets, rectangular in shape and varying size, using a charge model compared with one experimental measurement, N42 (5.1×5.1×5.1 cm) (blue solid line), N52 (10.2×7.6×5.1 cm) (red square), N52 (10.2×7.6×2.5 cm) (yellow square), experimental values for N42 (5.1×5.1×5.1 cm) (purple circle), B field required to open the capsule (15 mT) (green broken line).

FIG. 4 presents a graph depicting the maximum allowable angular misalignment of the external magnet which still results in the B field strength meeting the 15 mT minimum activation threshold to actuate the capsule to the open configuration, where the maximum angle error from nominal is plotted as a function of the external magnet misalignment. Below this maximum angle threshold, the field will still be above the minimum required 15 mT to open the capsule.

FIG. 5 is a perspective view of a safety box for the safe-handling of an external magnet, the magnet held in the centre of the box surrounded by open space.

FIG. 6 is a schematic illustration of the fabrication of the halves of the capsule shown in FIG. 1, (b, g) the capsule end molding structure; (c, h) carbon fiber mounting within the mold; (d, i) internal magnet installation; (e) pouring of silicone rubber; and (f, j) curing of the capsule sides.

FIG. 7 is a schematic illustration of (a) the process preparing the sealing mechanism of the capsule shown in FIG. 1; (b) mounting of carbon fiber in the hinge fixture and pouring of silicone rubber; and (c) mounting of the capsule in the hinge structure and curing of the hinge.

FIG. 8 presents a graph depicting the B field strength required to actuate the capsule shown in FIG. 1 to reconfigure to the open configuration where opening angle was measured against the applied field for five capsules. The dashed line indicates the theoretical results derived from solving of the torque equilibrium equation.

FIG. 9 presents photographs showing the results of sealing (a-c) and activation (d-f) tests, (a) inspecting capsule emptiness before insertion into the intestine section of proximal jejunum of a pig for the sealing test; (b) mechanical agitation of the intestine with the capsule inside; and (c) opening of the capsule after transit; (d) inspecting capsule emptiness before insertion into the intestine for the activation test, (e) capsule activation with a 5.1 cm cubic magnet, and (f) inspection of digestive tract sample collection by one representative capsule.

FIG. 10A to 10D present photographs showing self-alignment of a capsule to an applied field. FIG. 10A shows somersaulting action; FIG. 10B shows the capsule in a closed configuration (top photograph), and activation into the open configuration; FIG. 10C shows swiveling action; FIG. 10D shows motion of a capsule within a tilted tube filled with water (first and second photographs from the top), motion of the capsule being stopped (third photograph from the top) by use of an external magnet, and motion of the capsule being resumed when the external magnet is removed.

FIGS. 11A and 11B present photographs showing the sealing performance of a capsule, where the capsule is loaded with red dye to simulate a drug release scenario. FIG. 11A shows the slow release of the red dye by application of a small magnetic field, FIG. 11B shows quick release of the red dye by application of a larger magnetic field.

FIGS. 12A and 12B present graphs showing the approximate concentration of released dye over time during each release, corresponding to FIG. 11A and FIG. 11 B, respectively.

FIGS. 13A and 13B are illustrations of the use of a capsule according to an embodiment, as it passively transits through the intestinal tract of a human (FIG. 13A), and through the intestinal tract of a pig (FIG. 13B), and is activated by application of an external magnet.

### DETAILED DESCRIPTION OF THE INVENTION

The present invention provides for a tetherless, magnetically actuated capsule (MAC), and its use for noninvasive sampling and/or delivery of an agent anywhere along the gastrointestinal (GI) tract of a subject. The MAC is designed to be orally administered and swallowed by a subject to allow for the MAC to passively transit the GI tract where it can be remotely activated for sampling, or in certain embodiments delivery of an agent, by an external magnetic field. Samples are encapsulated and sealed in the MAC during passage through the GI tract and ultimately recovered via routine stool passage for further processing upon retrieval.

In certain embodiments, the MAC comprises a soft capsule body to permit noninvasive and safe passage, without cross-contamination, through the GI tract. According to certain embodiments, the capsule is sized to permit a subject to easily swallow and digest the MAC to permit its passage through all areas of the GI tract including, for example, typically inaccessible regions such as the small intestine. In certain embodiments, the MAC comprises materials that are compatible with being ingested, for example, materials that are safe, acid- and enzyme-resistant, and that do not inhibit cell viability of collected samples. The capsule materials, according to embodiments, remain intact while operating in the low-pH environment of the GI tract with various enzymatic activity.

According to embodiments, the MAC comprises an actuation mechanism that does not require an on-board motor or smart-materials (e.g., that responds to temperature or pH changes) to activate actuation of the MAC. In certain embodiments, the MAC is adapted to be remotely activated by external magnetic actuation. The MAC, in such embodiments, has a simplistic design comprising a minimal number of parts that can include, for example a capsule having two portions connected by a hinge wherein each portion contains a magnet, and a chamber for storing a sample during GI tract transit. In this way, embodiments of the present invention comprise minimal parts and are adapted to maximize the storage capacity of the MAC to permit larger volumes to be sampled and/or stored for delivery.

In certain embodiments, the MAC comprises materials having reinforced stiffness and/or strength at specific locations on the capsule body to reinforce the ruggedness of the capsule in high fatigue zones while ensuring a tight seal between the two cooperating portions of the capsule. According to embodiments, the MAC is adapted to tolerate various external and internal forces and torques during ingestion, GI transit and collection after passage in the feces. In such embodiments, the capsule body and hinge comprise a reinforced composite material to retain the integrity of the MAC in harsh environments and to withstand the force required for sealing, and actuation of the capsule between the open and closed positions. In particular, the composite material is reinforced at the hinge to tolerate the force and torque applied for capsule opening, and further facilitate capsule closure when the external field is removed. In such embodiments, the hinge composite has a stiffness sufficient to prevent the capsule from opening beyond its pivot point, i.e., to the point where the internal magnets stick together in the fully ‘folded-back’ open configuration.

In certain embodiments, the MAC is adapted to capture a sample from the GI tract and contain the sample within the capsule during capsule transition through the GI tract. According to certain embodiments, the internal chamber of the capsule comprises a hydrophilic coating to enhance the ability of the capsule chamber to capture sample during activation of the MAC. In certain embodiments, the capsule further comprises sealing means disposed between the two cooperating portions to prevent sample leakage and cross-contamination of collected samples with other liquids.

In certain embodiments, the present invention provides for the use of a MAC for the noninvasive retrieval of a sample, or in certain embodiments delivery of an agent, in the gastrointestinal (GI) tract. According to certain embodiments, the MAC can be remotely oriented by external magnetic actuation, which permits sample collection even when its location and orientation are not precisely known, i.e., the MAC can be operated blind in the GI tract.

In some embodiments, the invention provides for the simultaneous, noninvasive, sampling and/or delivery of agents at multiple locations within the GI tract. For example, certain embodiments provide for the use of a plurality of MACs administered to a subject at various time points and simultaneously activated to collect samples and/or delivery agents at various locations in the GI tract. The capsules are then recovered via routine stool passage and processed upon retrieval for downstream analysis of microbiome composition and/or function (e.g. 16S rRNA surveys, metagenomics, metatranscriptomics and/or metabolomics).

### Definitions

Unless defined otherwise, all technical and scientific terms used herein have the same meaning as commonly understood by one of ordinary skill in the art to which this invention belongs.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth. The term “plurality” as used herein means “one or more.”

Throughout this specification and the claims, the terms “comprise,” “comprises,” and “comprising” are used in a non-exclusive sense, except where the context requires otherwise. Likewise, the terms “include”, “has” and their grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter.

As used herein, the term “about” refers to an approximately +/−10% variation from a given value. It is to be understood that such a variation is always included in any given value provided herein, whether or not it is specifically referred to.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

As used herein, the term “tethered”, in reference to a device, refers to a device that has a cable or cord attached to it. This cable/cord is used to transfer power and control signals to the device. A considerable number of robots at the meter-scale are “tethered”. “Untethered” is a synonym of “wireless”. In microrobotics the word “untethered”/“tetherless” is preferred over “wireless”.

As used herein, the term “agent” includes both biologics and non-biologic agents. The biologic agents, or biopharmaceuticals, described herein are used for diagnosis, prevention and treatment of diseases or conditions. Biologic agents include, but are not limited to, antibiotics, fecal matter, nutritional agents, polyclonal/monoclonal antibodies or fragments thereof, recombinant proteins, enzymes RNAi, aptamers, and dendrimers, RNA, and DNA, cytokines, tissue growth factors, gene transfer products, glycosaminoglycans, cells (including stem cells), genetically modified cells (including genetically modified stem cells), bacteria, genetically modified bacteria, probiotics, live biotherapeutic products (LBP), viruses, genetically modified viruses, nutritional agents and so forth. The non-biologic agents described herein include, but not limited to, minerals, vitamins and small organic molecules used for diagnosis, prevention and/or treatment of diseases or conditions.

As used herein, the term “subject,” “individual” or “patient”, is used interchangeably herein, and refers to a vertebrate, preferably a mammal, more preferably a human. Mammals include, but are not limited to, murines, rats, rabbits, simians, bovines, ovines, porcines, canines, felines, farm animals, sport animals, pets, equines, and primates, particularly humans.

### Magnetically Actuated Capsule (MAC)

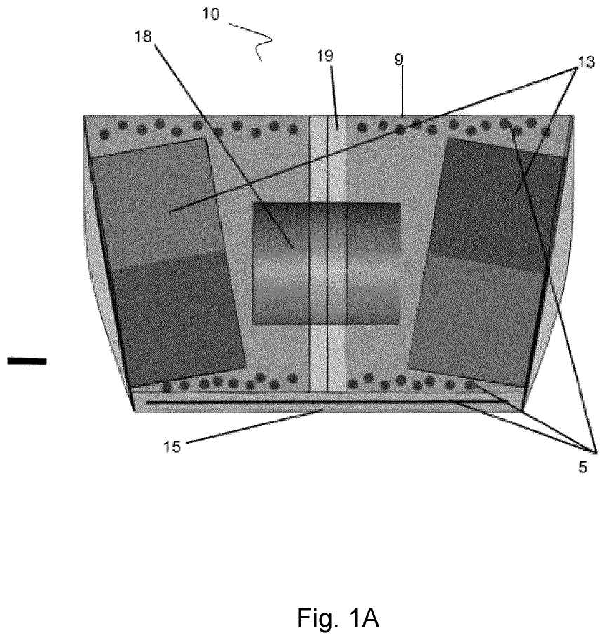

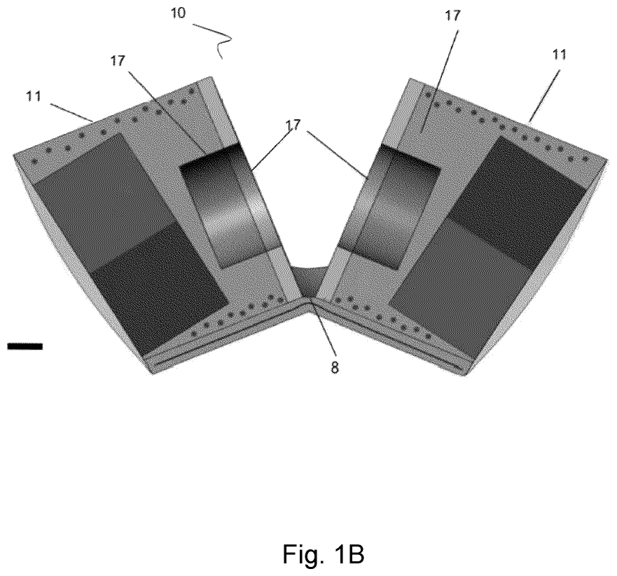

With reference to FIG. 1, a three-dimensional (3D) untethered mobile actuator 10, also referred to as “capsule” or “MAC” (terms to be used interchangeably herein), has two cooperating portions 11 that together enclose an interior chamber 18. Each portion 11 of the MAC 10 is sized to house at least one magnet 13. In certain embodiments, the MAC 10 comprises a pair of magnets 13, one magnet 13 in each portion 11. In other embodiments, the MAC 10 may comprise more than one magnet 13 in each portion 11, for example, 2, 3, or 4, magnets 13 in each portion 11. In another embodiment, permanent magnetic particles may be embedded in each portion 11 of the capsule 10.

The two portions 11 of the capsule 10 are connected by a hinge 15, in certain embodiments, to allow the portions 11 to pivotally move between a closed configuration (FIG. 1A) and an open configuration (FIG. 1B) about a pivot point 8.

When in the closed configuration, the two portions 11 form a closed chamber 18 within the capsule 10 for storing a sample or agent for delivery. According to certain embodiments, the portions 11 may be unequal in size such that the chamber 18 is formed within one of the portions 11 and the other portion 11 forms a lid or wall for closing the chamber 18 when the capsule 10 is in the closed configuration. In another embodiment, the portions 11 are about equal in size such that the chamber 18 extends between both portions 11 as shown in FIG. 1A. When the capsule 10 is actuated into the open configuration, the two portions 11 move apart from each other at the pivot point 8 to define an aperture 17 exposing the chamber 18 to the exterior of the capsule 10 as shown in FIG. 1B.

According to certain embodiments, the chamber 18 can be treated to further enhance sample capture. The volume of sample collected can be affected in part by the affinity that the sample to be collected has to the inner surface of the capsule chamber 18. In certain embodiments, the surface energy of the chamber 18 can be increased to maximize this affinity and increase the collected sample size. In such embodiments, the surface of the chamber 18 can be treated with a hydrophilic coating to increase the surface energy of the chamber 18. According to embodiments, the hydrophilic coating is a biocompatible coating that improves cell adhesion to silicone rubber surfaces. In particular embodiments, the hydrophilic coating on the silicone rubber surface of the chamber 18 is polydopamine.

Retaining the contents of the chamber 18 during transit through the body cavity is necessary to avoid cross-contamination of collected samples and/or leaking of agents contained in the chamber 18 for delivery. According to certain embodiments, the portions 11 of the MAC 10 have a seal 19 to ensure that the capsule 10 remains closed and liquid impermeable before and after activation, so as to prevent leakage into and out of the chamber 18, thereby preventing cross-contamination with other liquids during capsule 10 transition through the GI tract under conditions such as agitation and irregular motion. In certain embodiments, the exterior surface of the capsule 10 comprises a seal 19 at the aperture-defining end of each portion 11. The seal 19 in such embodiments is liquid tight to maintain closure of the MAC 10 in the closed configuration and to prevent the contents of the chamber 18 from leaking out or into the chamber 18. According to certain embodiments, the seal 19 comprises silicone rubber having sufficient adhesion force to avoid leakage and sample contamination. In certain embodiments, the seal 19 comprises a silicone rubber that is more compliant than that used in the capsule 10 body in order to create a liquid tight seal between the capsule portions 11.

According to embodiments, the capsule 10 is sized and shaped to facilitate transit through a subject's body cavity, for example, the gastrointestinal (GI) tract. In certain embodiments, the capsule 10 is cylindrical in shape. In other embodiments, the capsule 10 is approximately semi-cylindrical or semi-circular in shape. Each of the two portions 11 can be symmetrical in certain embodiments, or alternatively, the two portions 11 may differ in shape. In certain embodiments, for example, the capsule 10 may comprise a first portion 11 having a semi-hemispherical shape and a bluntly closed second portion 11. In one aspect, the two portions 11 are symmetrically semi-hemispherical in shape as shown in FIG. 1A.

According to embodiments, the capsule 10 is sized to accommodate the intended body cavity environment and the size of the intended subject. In certain embodiments, the capsule 10 is sized for use in the GI tract, for example. In such embodiments, the capsule 10 is cylindrical in shape having an outer diameter of about 5 mm, 6 mm, 7 mm, 8 mm, 9 mm, 10 mm, 11 mm, or 12 mm; and a length of about 7 mm, 8 mm, 9 mm, 10 mm, 11 mm, 12 mm, 13 mm, 14 mm, or 15 mm. In other embodiments, the capsule 10 has an outer diameter of between about 6 mm to about 10 mm and a length of between about 9 mm and about 13 mm. In other embodiments, the capsule 10 has an outer diameter of about 8 mm and a length of about 11 mm.

The size and shape of the chamber 18 is determined by the dimensions of the capsule 10. According to embodiments, the chamber 18 has a volume of about 15 μL, 18 μL, 20 μL, 23 μL, 25 μL, 27 μL, 30 μL, 32 μL, 35 μL, 37 μL, 40 μL, 42 μL, 45 μL, 48 μL, or 50 μL. In other embodiments, the chamber 18 has a volume that ranges between about 20 μL to about 45 μL. In other embodiments, the chamber 18 has a volume that ranges between 25 μL to about 40 μL. In further embodiments, the chamber 18 has a volume that ranges between about 28.9 μL to about 38.5 μL.

The MAC 10 is constructed with materials that tolerate the various internal and external forces and torques that the MAC 10 may encounter. In the case of the GI tract, for example, the entire MAC 10 has to tolerate the internal/external forces and torques during ingestion, GI transit and collection after passage in the feces. The construction material should not collapse under the attraction forces used for sealing and must be safe to the subject ingesting the actuator 10. As such, in one embodiment, the capsule 10, with the exception of the magnets 13, is soft yet resilient enough to ensure that the capsule 10 does not collapse under deformation, that the magnets are held securely in place, and to reinforce the ruggedness of high fatigue elements of the capsule 10 such as the capsule portions 11 and the hinge 15.

In addition to material strength, the materials forming the capsule 10 and coming into contact with tissue of the subject must be safe and compatible with such purposes. In cases such as the GI tract, for example, compatibility means that the materials forming the capsule 10 are acid- and enzyme-resistant, and do not inhibit or affect cell viability of collected samples. Additionally, the capsule 10 materials must remain intact while operating in the low-pH environment of the GI-tract with various enzymatic activity.

According to embodiments, the two portions 11 of the capsule 10 can be made of a polymeric elastomer material known in the art. In certain embodiments, the capsule 10 comprises a silicone rubber composite. The strength of the material can be reinforced, in certain embodiments, to meet the performance requirements of the particular application. In such embodiments, a silicone rubber carbon fiber composite 5 is used to reinforce the capsule body as shown in FIG. 1A.

The hinge 15, in particular, is the most vulnerable part of the MAC 10 as it must tolerate the force and torque applied to the capsule 10 for actuation to the open configuration. The hinge 15 also contributes to capsule 10 closure when the external field is removed. In this way, the hinge 15 is a high fatigue zone of the capsule 10. According to certain embodiments, the hinge 15 is reinforced to provide sufficient strength for operability in such high fatigue performance. In particular, the hinge 15 comprises a composite material that has a stiffness sufficient to prevent the capsule from opening beyond its pivot point 8, i.e., to the point where the internal magnets stick together in the fully ‘folded-back’ open configuration. The stiffness of the hinge 15 is also sufficient to withstand, without breaking, the application of a large external magnetic field to activate the MAC 10 for opening. In certain embodiments, the hinge 15 is reinforced with a silicone rubber carbon fiber composite.

### Tetherless Remote Actuation Mechanism

The MAC 10 is an autonomous, untethered capsule 10 which can be opened and closed remotely simply by toggling an external magnetic field, which is useful for remotely collecting a sample or releasing a cargo in a body cavity. The capsule's 10 position, orientation, reconfiguration in the open and closed configuration, and delivery actions are controlled autonomously and independently. In accordance with embodiments, the MAC 10 can be fully controlled by an external magnetic field, without the need for other forms of input such as thermal, optical, electrical or chemical inputs that are typically used in prior art devices.

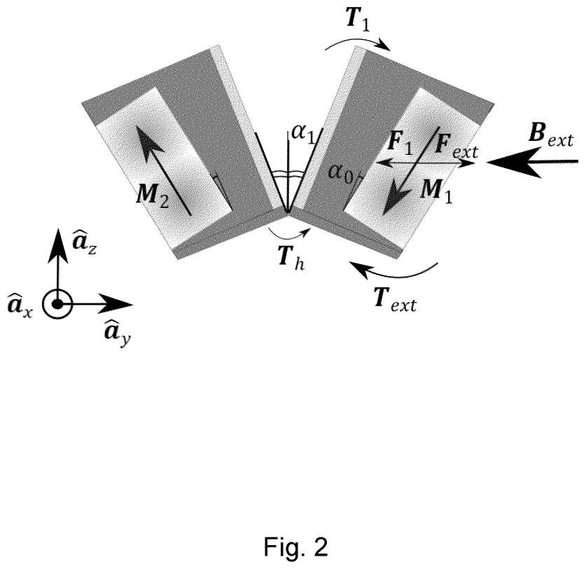

In certain embodiments, the capsule 10 is in the closed configuration (FIG. 1A) until it is activated to reconfigure into the open configuration by application of an external magnetic field (FIG. 1B). As illustrated in FIG. 2, this applied magnetic field Bₑₓₜ generates a torque on each internal magnet 13, housed in the respective portion 11 of the capsule 10, to bring the capsule 10 into alignment with the field. The effect of the applied field is thus to create an opposite magnetic torque on each capsule magnet 13, to reconfigure the capsule 10 into the open configuration.

According to certain embodiments, the magnets 13 housed in each portion 11 are identical and disposed within the respective portion 11 in opposite magnetization direction (M₁, M₂) from the other magnet 13. In other words, the magnets 13 are disposed in the respective portion 11 such that their magnetic moment (M₁, M₂) is in a direction opposite to the magnetic moment (M₁, M₂) of the other magnet 13 in the other portion 11.

According to certain embodiments, the magnets 13 are permanent magnets or “hard” magnets that retain their magnetization once a strong magnetizing field is applied and removed. In this way, the incorporation of permanent magnets into cooperating portions 11 of the capsule 10 allows the capsule 10 to be magnetically “programmed” for remote tetherless actuation. In certain embodiments, the MAC 10 comprises a pair of magnets 13, one magnet 13 in each portion 11. In other embodiments, the MAC 10 may comprise more than one magnet 13 in each portion 11, for example, 2, 3, or 4, magnets 13 in each portion 11. In another embodiment, permanent magnetic particles may be embedded in the shell of the MAC 10.

In one embodiment, the two permanent magnets 13 of the MAC 10 may be disk magnets having opposite magnetization direction from one another, the magnetization being in a direction along a diameter of each internal permanent magnet 13. The orientation of the magnets 13, in the respective portion 11, in opposite magnetization directions generates an inter-magnet attraction force between the permanent magnets 13 that maintains the capsule 10 in the closed configuration in the absence of an externally applied magnetic field. Moreover, in certain embodiments, the magnitude of this attraction force is sufficient to create a liquid tight seal between the portions 11 for leak-proof transit of the capsule 10 while still allowing opening of the capsule 10 with application of a minimal requisite external magnetic field.

Magnetic actuation of the MAC 10, according to embodiments, requires the external magnetic field to be applied in the correct direction to open the MAC 10. In certain embodiments, the MAC 10 comprises a small net magnetic moment to allow the entire capsule 10 body to self-align with the applied field Bₑₓₜ into the correct heading. In such embodiments, this slight magnetic moment can be created by introducing a small angle α₀ in the orientation of the internal magnets 13 in the respective portions 11 as shown in FIG. 2. In certain embodiments, the angle α₀ is between about 5° and about 30°. In other embodiments the angle α₀ is 5° or 5°±10%, 10° or 10°±10%, 15° or 15°±10%, 20° or 20°±10%, 25° or 25°±10%, 30° or 30°±10%. In one embodiment the angle α₀ is 10°.

Referring to FIG. 2, the magnetic actuation mechanism of the MAC 10 is activated by application of an external magnetic field Bₑₓₜ which creates opposing magnetic torques Tₑₓₜ on each of the opposing magnets 11. This torque balances with the hinge torque Tₕ and the interaction force and torque between the two magnets F₁ and T₁.

The external magnetic field Bₑₓₜ can be generated by a large external permanent magnet, or several stacked or parallel magnets [29][30] to activate the capsule, depending on the size of the subject. According to certain embodiments, one or more external magnet(s) of a size and weight that can be manually handled is used. In such embodiments, the one or more external magnet(s) is approximately 1 kg, 1.5 kg, 2 kg, 2.5 kg, or 3 kg in weight. According to other embodiments, the one or more external magnet(s) is an electromagnet to offer higher precision MAC 10 activation.

According to embodiments, the actuation mechanism of the MAC 10 can be activated blindly. Persons of skill in the art will understand that blind activation means magnetically actuating reconfiguration of the capsule 10 into the open configuration such that the aperture 17 is sufficiently wide to blindly (i.e., when the capsule has an unknown location and orientation) collect a sample in the chamber 18, or blindly deliver an agent or object into the subject's body cavity. In certain embodiments, the MAC 10 is blindly activated in a subject's body cavity, for example, a subject's GI tract, without the use of any localization system. According to embodiments, the MAC 10 is blindly activated by exposing the entire body of a subject to a magnetic field, by moving an external permanent magnet over the body, over the duration of activation.

### Selection and Placement of Internal and External Magnets

Operation of the actuation mechanism for MAC 10 functionality requires control of the forces and torques involved in activating the reconfiguration of the MAC 10 between the open and closed configurations. The overall design of the MAC system, including the selection and placement of internal and external magnets, will be dependent on the environmental context for use of the MAC 10, for example, the size of the subject and the body cavity targeted. According to embodiments, an analytical model of capsule opening can be used to inform for proper sealing and blind opening as well as geometric design of the capsule hinge, as exemplified herein in the context of the GI tract for purposes of illustration which is not intended to limit the scope of the invention.

### A. Model for the Internal Magnets

The torque required for magnetic opening of a MAC, scales with the volume of the internal magnets. Thus, the largest internal magnet volume (considering GI tract limitations) and highest grade are preferably selected to generate a strong internal magnetic moment.

With reference to FIG. 2, the internal magnets can be approximated by a dipole magnetic moment of m₁ for the right side and m₂ for the left side. This magnetic moment is a vector pointing from the south to north magnetic pole of the internal magnet proportional to the magnet strength. Relevant magnetization vectors (M₁ and M₂) are labeled on the capsule free body diagram (FIG. 2), and m₁=M₁V and m₂=M₂V, where V is the magnet volume, are equal for both internal magnets. Each internal magnet will experience a torque and force generated by the other internal magnet as well as an activating torque and force generated by the externally-applied magnetic field. The torque on an internal magnet m due to an applied field B is m×B, which acts to bring the magnetic moment into alignment with the field. The magnetic force on an internal magnet is created by a field gradient and is (m·∇)B, which acts approximately to move magnets towards regions of higher field strength.

The internal torque and force applied to m₁ by the other magnet m₂ are T₁ and F₁, respectively. The activating torque and force on m₁ by the external field Bₑₓₜ are Tₑₓₜ and Fₑₓₜ, respectively. The hinge also generates a resistive torque for capsule opening [symbol] ₕ. These forces and torques on the capsule half sum and are balanced at a certain capsule opening angle α according to the torque balance equation

T₁+L×F₁+τₕ+m₁×Bₑₓₜ=0 (1)

where L is the position vector originating from the hinge center of mass (COM) to the internal magnet COM (Magnet 1). The total angle α is the sum of the small offset angle α₀ and half of opening angle α₁. To calculate the field and field gradient generated by the internal magnet for use in (1), a dipole field model is used.

$${T}_{1} = - \frac{{μ}_{0} {m}^{2}}{8 π {r}^{3}} sin ( 2 α ) {\hat{a}}_{x} and \qquad ( 2 )$$

$${F}_{1} = \frac{3 {μ}_{0} {m}^{2}}{4 π {r}^{4}} ( - 1 - {sin}^{2} ( α ) ) {\hat{a}}_{y ′} \qquad ( 3 )$$

where μ₀ is the magnetic permeability of free space (1.257×10⁶ H/m), m=m₁=m₂, r is the distance between the COMs of the internal magnets, and âₓ and â_{y} are the unit vectors of the coordinate system indicated in FIG. 2.

Therefore, Equation 1 can be expressed as follows:

$$\frac{{μ}_{0} {m}^{2}}{8 π {r}^{3}} sin ( 2 α ) {\hat{a}}_{x} + \frac{3 {μ}_{0} {Lm}^{2}}{4 π {r}^{4}} ( 1 + {sin}^{2} α ) cos ( {α}^{′} + {α}_{1} ) {\hat{a}}_{x} + ( \frac{3 EI tan ( {α}_{1} )}{{l}^{′}} ) {\hat{a}}_{x} - {mB}_{ext} cos ( α ) {\hat{a}}_{x} = 0 , \qquad ( 4 )$$

where r is the distance between the COMs of the internal magnets, and α is the angle between the plane intersecting both halves of the capsule and the plane passing through the COMs of the hinge and internal magnet across the capsule diagonally. The hinge torque (τₕ) is approximated with the elastic model of a rectangular cantilever beam using the Euler-Bernoulli theory, where l′ is the length of the hinge, E is the Young's modulus calculated experimentally from the results of the tensile test, and I is the second moment of area. The hinge geometrical properties (e.g., thickness and length) play critical roles in Equation 4.

In certain embodiments, based on this model, the placement of the magnet 13 within each half of the capsule 10 contributes to the control of the opening and closing of the MAC 10 and is parameterized by the position vector L. In such embodiments, the position vector L is selected to provide sufficient sealing force while maintaining the ability to open the capsule under the available magnetic field from the external magnet.

### B. Model for the External Magnetic Field

The external magnetic field required to open a MAC 10 will have a minimum threshold that is affected by the sealing adhesion, inter-magnet attraction, and the hinge torque exhibited by the capsule 10. According to embodiments, a charge model can be used to determine the appropriate external magnetic field to operate an actuation mechanism, as exemplified herein.

In this model, the permanent magnet is modeled with a distribution of volume (ρₘ) and surface (σₘ) magnetic charges. The volume and surface charge densities are calculated as ρₘ=−∇·M and σₘ=M·^{\hat{n}}, respectively, where M is a magnetization vector and \hat{n} is a surface normal vector. Due to uniform polarization, M=M_{S}â_{z}, thus ρₘ=−∇·M=0 and the model is simplified to a surface charge distribution [32]. The B field along the central axis (z) of the magnet is calculated as [32]:

$$B ( z ) = \frac{{μ}_{0} {M}_{S}}{π} [ {tan}^{- 1} ( \frac{( z + L ) \sqrt{{a}^{2} + {b}^{2} + {( z + L )}^{2}}}{ab} ) - {tan}^{- 1} ( \frac{z \sqrt{{a}^{2} + {b}^{2} + {z}^{2}}}{ab} ) ] , \qquad ( 5 )$$

where a and b are the dimensions of the front surface and L is the thickness of a magnet bar.

### Fabrication of MAC

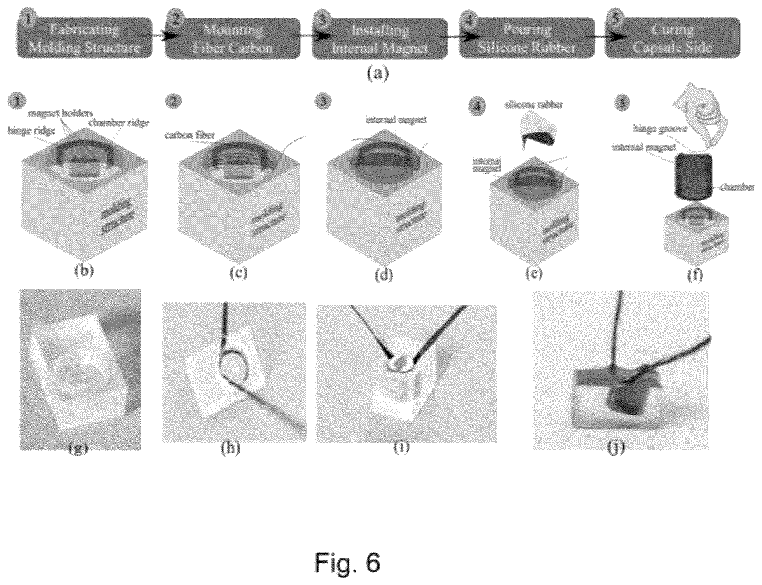

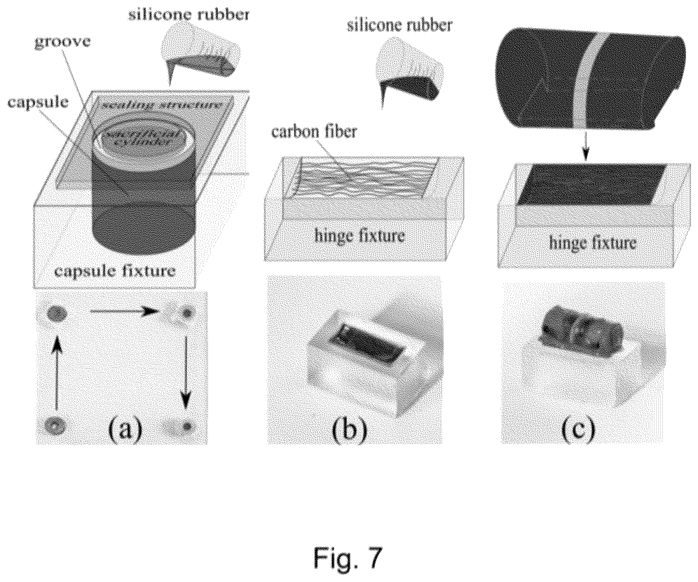

FIGS. 6 and 7 show the fabrication process of a MAC 10 according to one embodiment of the present invention. The fabrication of the capsule 10 of the present invention includes, in one embodiment, the following steps.

(a) providing molds for each of the capsule's portions 11, each mold including a chamber ridge;
(b) mounting one or more permanent magnets 13 in each mold, the permanent magnets 13 are positioned in the mold of each portion 11 such that the two portions 11 are biased to magnetically bind to each other to form the capsule 10;
(c) pouring a polymer into each of the molds to form the portions having the permanent magnets 13;
(e) contacting the two portions 11 at a rim of the openings of each portion 11 to form the capsule 10, and placing the capsule 10 in the hinge mold having the composite such as only one side of the capsule 10 contacts the composite, thereby producing the capsule 10 having the hinge 15 on that one side.

The mold of each portion 11 in one embodiment includes a magnet holder designed to orient each permanent magnet 13, and the permanent magnets 13 are mounted to the magnet holders (see FIG. 7). The one or more permanent magnets 13 are oriented in the mold of each portion 11 such that in the formed capsule 10 a flat circular surface of the permanent magnet 13 will be at an angle αₒ relative to the interface plane and the point closest to the interface plane on the flat circular surface will also be the point on the flat circular surface closest to the other permanent magnet 13.

According to embodiments, the method further comprises creating a seal groove around the ridge of the opening of each portion 11, and pouring a sealing composite into the seal groove, as illustrated in FIG. 7.

As further illustrated in FIG. 7, the seal groove is formed by inserting a sacrificial body inside the chamber 18 of each portion 11 and a sealing structure that includes a hole that matches a rim of an outer surface of each portion 11 so as to form the seal groove around the rim of the opening of each portion 11.

### Methods and Uses

The present invention provides for the use of a magnetically actuated capsule for collecting physical samples from a body cavity of a subject and/or delivering agents to a targeted location in a body cavity of a subject. In one embodiment, the MAC can be used to collect samples for the diagnosis or treatment of a disease or condition associated with said physical sample. Such an approach is useful for example, to diagnose a disease or condition associated with the GI tract.

Certain embodiments provide for the collection of samples from the body cavity of a subject for the downstream analysis of samples. Such an application is useful, for example, in the analysis of microbiome composition and/or function (e.g. 16S rRNA surveys, metagenomics, metatranscriptomics and/or metabolomics) in samples collected from the GI tract. According to certain embodiments, the sample collected is a liquid digesta microbiome sample.

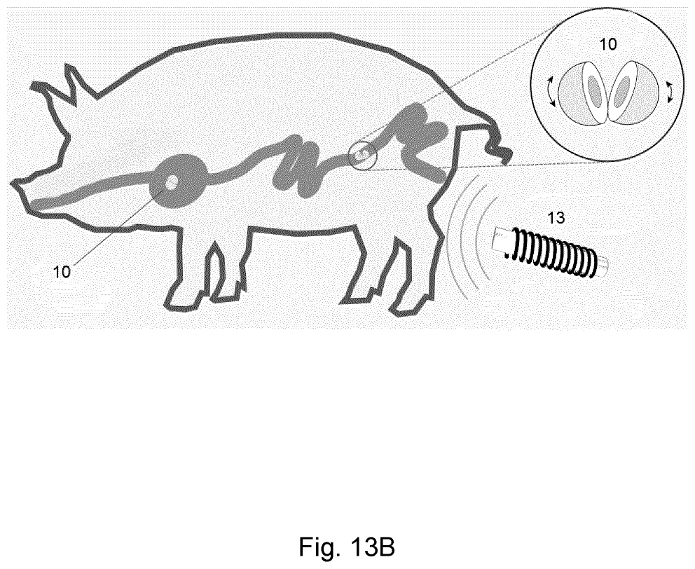

Referring to FIGS. 13A and 13B, according to one embodiment for collecting samples, a capsule 10 in the closed configuration is provided to a subject for ingestion by the subject. Time is allowed to elapse for the capsule to passively transit through the subject's intestinal tract for a predetermined period of time. An external magnetic field 13 is then applied to the subject to actuate reconfiguration of the capsule 10 to the open configuration, thereby exposing the chamber 18 of the capsule 10 to the exterior of the capsule 10 for collection of the sample in the chamber 18. The external magnetic field 13 is then removed to actuate reconfiguration of the capsule 10 to the closed configuration, thereby enclosing the sample in the chamber 18. The capsule 10 is then allowed to transit through the intestinal tract for recovery by stool passage.

Certain embodiments provide for the delivery of objects, cargoes or active biological or non-biological agents, inside a body cavity that could be used to treat a disease or condition, for example. In other embodiments, it is contemplated that the MAC can also be used to enlarge vessels, to open or remove blockages in a body cavity or vessel, and so forth.

In one embodiment, the capsule can be used to delivery drug payloads within a body cavity. The delivery can be done at a specific time and/or location within the body cavity, such as the GI tract. In this embodiment, a drug payload is pre-loaded into the capsule prior to oral administration of the capsule. The drug could take the form of a liquid, powder or gel, but would be released most quickly from the capsule at activation in liquid form. The capsule is administered orally. After waiting for the capsule to reach the targeted location in the GI tract for therapy (as confirmed by either elapsed time, capsule magnetic tracking, or medical imaging) the capsule is opened wirelessly by an externally applied magnetic field by an external permanent magnet or electromagnet. The field is applied for an effective amount of time to allow the drug time to exit the capsule by mixing with GI tract contents, such as for about 2-10 seconds. To promote better mixing of the drug to exit the capsule, the capsule is oscillated back-and-forth angularly by rotating the external magnet. After applying the external magnetic field for the effective amount of time, the external magnetic field is removed which allows the capsule to close back up. After the drug has been released, the capsule is allowed to passively pass through the GI tract and excreted. While there is no need to collect the capsule afterwards, the passage should be noted or followed to ensure that the capsule has exited the GI tract.

According to certain embodiments, multiple capsules can be used together to simultaneously sample and/or deliver at multiple locations in the body cavity. In such embodiments, the capsules are ingested by the subject in intervals to avoid the capsules clumping together in the body cavity, such as the stomach, as they will attract each other magnetically. In certain embodiments, the capsules are ingested by the subject no sooner than about 45 minute intervals. In other embodiments, the capsules are ingested in about 45 minutes, 60 minutes, 90 minutes, 120 minutes, or 180 minutes intervals. In further embodiments, the capsules are ingested in intervals over a course of no more than between about 3 hours and 24 hours.

Although the description and examples concentrate on the application of the magnetically actuated capsule for collecting samples from the GI tract scenario, persons of skill in the art would comprehend these and other alternative applications of the magnetically actuated capsule as a natural extension of the present invention. For example, in one embodiment, the magnetically actuated capsule may also be used to deliver a cargo, such as an object or an agent like a pharmaceutical agent, to the GI tract of an animal or to an animal cavity, including a cranial cavity, a vertebral cavity, a thoracic cavity, an abdominal cavity, a cardiac chamber and a pelvic cavity. In embodiments, the magnetically actuated capsule may also be used to collect samples and/or deliver cargoes in cavities other than a cavity of an animal.

To gain a better understanding of the invention described herein, the following examples are set forth. It will be understood that these examples are intended to describe illustrative embodiments of the invention and are not intended to limit the scope of the invention in any way.

### EXAMPLES

### Example 1: Fabrication of a Magnetically Actuated Capsule

This example describes a process for fabricating a magnetically actuated capsule (MAC). The MAC is designed as a cylindrical silicone rubber composite capsule having an outer diameter of 8 mm and a length of 11 mm. Parameters for fabrication were set pursuant to analytical modeling methods described herein. Each of the two sides of the MAC contains a disk magnet, and a cylindrical chamber (28.9-38.5 μL volume) for sample storage during GI tract transit.

The capsule's permanent magnets (type N52; 0.6 cm diameter, 0.3 cm thickness; K&J Magnetics Inc.) were magnetized in the direction of their diameter and mounted in the capsule. The magnitude of magnetic moment was determined as approximately 0.124 Am². This value was evaluated by measuring the magnetic field versus distance and fitting a dipole model to approximate the magnitude of the magnetic moment. This value is very close to reported one by the manufacturer as 0.1178 Am².

In three steps, the separate capsule sides, sealing mechanism, and hinge are fabricated. The three pieces are then assembled. The process is summarized in schematic flow chart presented in FIGS. 6 and 7.

### Capsule Side Fabrication

A molding structure consisting of a hinge ridge, chamber ridge (creating cavities for the respective components), and magnet holders was created as outlined in FIG. 6. A unidirectional carbon fabric (2585-A; Fibre Glast Developments Corp.) was wrapped around the chamber. Magnets were mounted on the holders, designed to orient the magnets at small offset angle α₀, in the mold. Here α₀ was chosen as 10° which is a compromise between capsule activation strength and net magnetic moment strength for capsule orientation control. During curing, the internal capsule magnets were held in the correct orientation by a magnetic field used as a fixture, generated by 2.5 cm cubic magnets (type N52; K&J Magnetics Inc.). The external magnet and mold were mounted on a physical fixture to maintain magnet directionality.

Silicone rubber (Mold Star 30, Smooth-On Inc.) was used at a 1:1 mass ratio of A and B parts from the manufacturer. The rubber was degassed in a vacuum chamber before and after pouring into the mold. The capsule sides were left overnight to cure.

### Sealing Mechanism Fabrication

On the chamber, three orifices caused by the attachment of the magnet to the magnet holders were repaired by injection of the same silicone rubber (Mold Star 30). Then, the prepared capsule sides were mounted on a fixture for seal fabrication (FIG. 7(a)). A sacrificial cylinder was inserted into the chamber to form the seal geometry precisely around the rim of the capsule halves. The sealing structure and sacrificial cylinder were cubic and cylindrical laser-cut acrylic sheets with dimensions of 10 mm and diameter of 4 mm, respectively.

Degassed Mold Star 30 (10:1 mass ratio of parts A to B) was poured into the groove between the sealing structure and sacrificial cylinder and cured for 12 h. Then, the cylinder and structure were removed from the capsule side for hinge preparation.

The silicone rubber for seal fabrication was more compliant than that used for the capsule sides. This lower stiffness rubber was created by using approximately 40% less curing catalyst, which in turn reduced the amount of crosslinking and resulted in less stiffness.

### Hinge Mechanism Fabrication

For fabrication of the MAC hinge, the composite was reinforced with carbon fibers (2585-A; Fibre Glast Developments Corp.). Before and after fiber mounting in the hinge fixture, Mold Star 30 (1:1 mass ratio of parts A to B) was poured into the fixture. Then, the attached capsule sides were mounted in the composite and the hinge was left overnight to cure (FIGS. 7 (b) and (c)).

### Internal Coating for Capture Enhancement

Dopamine hydrochloride (Sigma-Aldrich) was dissolved in Tris-HCl buffer (10 mM, pH 8.5 (adjusted by addition of 37% HCl) to a concentration of 2 g/L to create the polydopamine solution as in [28]. The polydopamine solution was then placed into both sides of the capsule chamber (20 μL each) for 24 hours under air. The chambers were then flushed and washed three times with deionized water for 10 min with sonication and dried at room temperature overnight.

### Example 2: Determination of the Required Magnetic Field and Minimum Capsule-External Magnet Distance for Activation

An in-vitro experiment was performed to determine the required magnetic field and minimum capsule-external magnet (type N52, 10.2×7.6×2.5 cm) distance for activation of the MAC described in Example 1. The magnet's magnetic field was measured five times at various external magnet-to-capsule distances to determine the field required for activation. This measurement was repeated for 12 different capsules. The average distance required for activation was 5.3 cm, with a 15-mT field required for activation (to overcome internal torques and the adhesion caused by the sealing surfaces of the two capsule halves).

### Example 3: Characterization of the External Magnetic Field

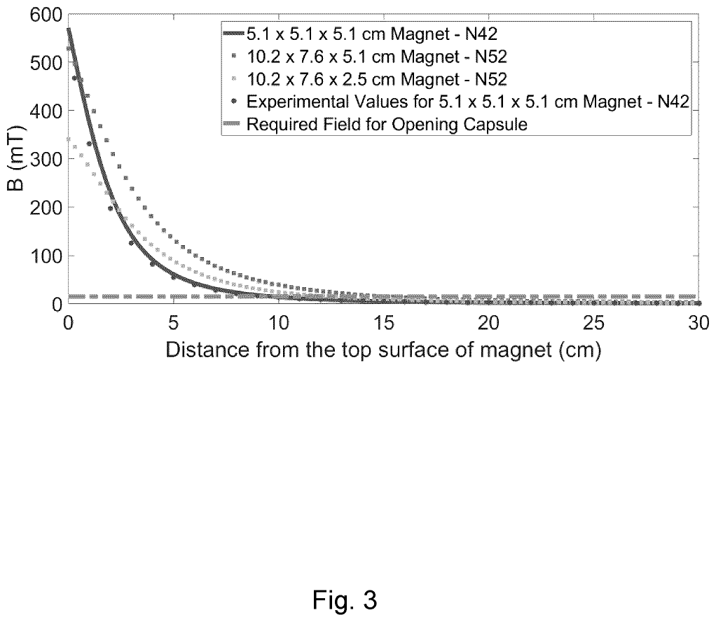

The B field generated by three different commercially available external magnet sizes were studied: N42 (5.1×5.1×5.1 cm), large N52 (10.2×7.6×5.1 cm), and intermediate N52 (10.2×7.6×2.5 cm) and compared to experimental values based on modeling methods described herein. The B field of each magnet was measured as a function of the distance (z) from the front surface of the magnet. The results are presented in FIG. 3.

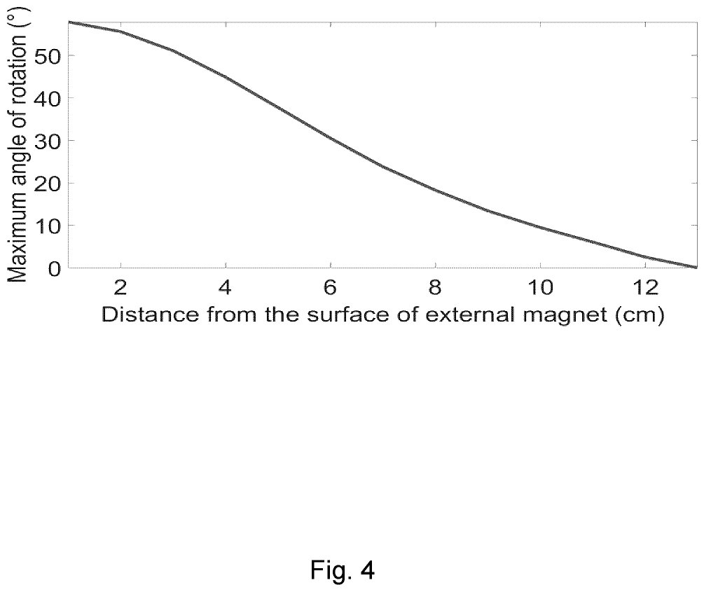

Because the external magnet is manually controlled by hand, the effect of small angular misalignments of the external magnet from the capsule's true location was also studied. A properly aligned external magnet points the external magnet directly at the capsule. However, during blind activation of the capsule, the external magnet will be scanned systematically over the entire abdomen but will likely not point directly at the capsule when at its closest approach point. This effect is shown in FIG. 4, where the maximum angle error from nominal is plotted as a function of the external magnet misalignment. Below this maximum angle threshold, the field will still be above the minimum required 15 mT to open the capsule.

From these results, the intermediate-size external magnet (type N52, 10.2×7.6×2.5 cm; K&J Magnetics Inc., magnetized along its thickness) was chosen because it is easier to handle than the largest type, and still generates a large enough field for capsule activation.

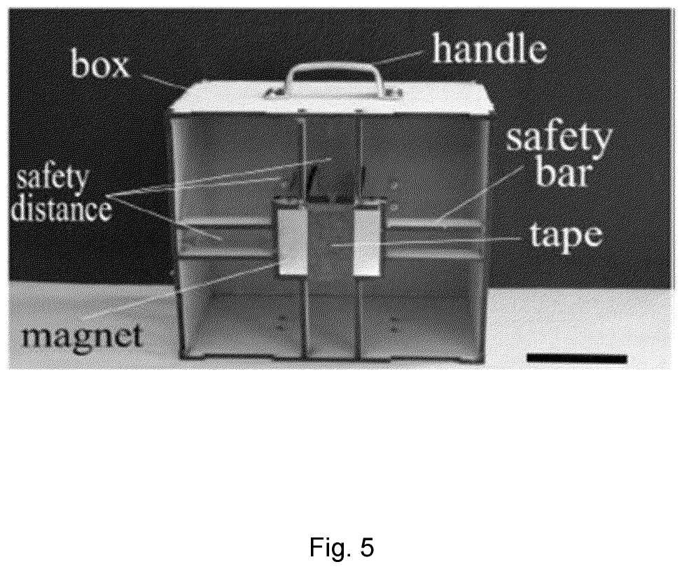

For safe handling of the external magnet during testing, the external magnet was secured within a magnet safety box or magnet holder (FIG. 5). As illustrated, the magnet is attached within the box to safety bars that keep the magnet at a center within the box at substantially equal safety distances from the sides of the box. The safety bars are vertically and horizontally disposed and tape can be used to attach the magnet to the safety bars.

### Example 4: Determination of Capsule Sealing

An MAC agitation experiment was performed to test the capsule sealing. 10 μL of green food dye was injected into the sampling chambers of five capsules. Each was immersed individually in a 10-mL tube filled with 50 mL deionized water. The tubes were then agitated in a centrifuge (Sorvall Legend X1R; Thermo Scientific) at 300 rpm for 1 h at 25° C. Then, they were vortexed at maximum speed for 5 min in an analog vortex mixer (VWR International, LLC). To test how much green food dye escaped the capsule during this agitation, a calibration curve of five known dye concentrations (0.02, 0.01, 0.0075, 0.005, and 0 vol %) was made and the average concentration of food dye in the water of vortexed capsules was determined by UV-vis spectroscopy (Cary 50 UV-Vis spectrophotometer, Agilent Technologies). The seal was effective in preventing major leakages of dye out of the capsule (0.001±0.0004 vol % dye in water after vortexing, n=5).

### Example 5: Determination of Capsule Self-Alignment

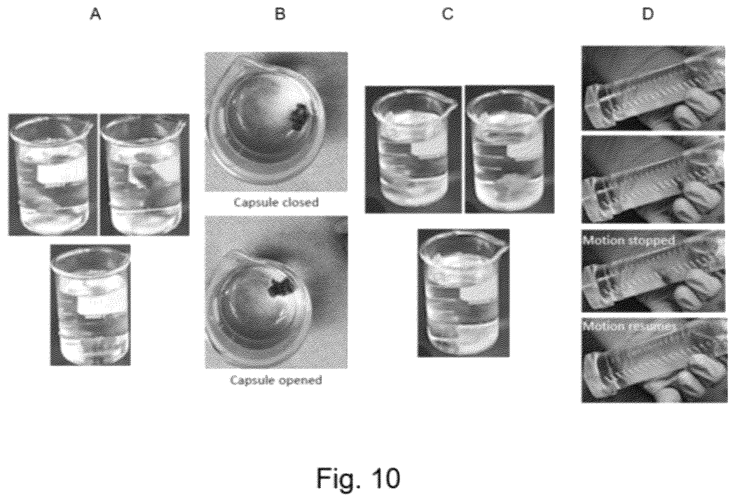

The capsule self-alignment to an applied field was tested in an acrylic tub container. MAC summersaulting (FIG. 10A), orienting, activating (FIG. 10B), swiveling (FIG. 10C) and stopping (FIG. 10D) actions were tested by moving an external magnet around a beaker in which the capsule was immersed in water, with a resulting motion.

### Example 6: Determination of the Opening Angle

To experimentally measure the opening angle of the capsule under different external magnetic field strengths, fields were applied in a Helmholtz electromagnetic coil set. In this coil pair, precise coil currents are applied by three analog servo drives (30A8; Advanced Motion Controls). Custom control code was used with a multifunction analog/digital I/O board (model 826; Sensoray) to control the coil current. Coil calibration was performed with a gaussmeter (LakeShore Cryotronics). Two cameras (FO134TC; FOculus) at the coil side and top were used at 30 frames/second to provide a view of the capsule opening motion. The coil system was able to generate a uniform field up to 18 mT in arbitrary three-dimensional directions near its geometric center, with ±5% error within a 44 mm sphere.

Each of five capsules was placed in the center of the coil. Top-view images were obtained in parallel projection (i.e., parallel to the lateral capsule view) for capsule opening angle measurement. Side-view images were used to indicate the capsule position to ensure the MAC was located in the center of the coil's workspace to ensure field uniformity. The magnetic field was applied along the capsule long axis. The capsules moved freely in contact with the bottom of the coil's working space, permitting symmetrical opening of both sides. To avoid surface tension and adhesion caused by the soft capsule seal during the opening time of the capsules, the magnetic field was first applied at 18 mT to fully open the capsule and then decreased to near zero.

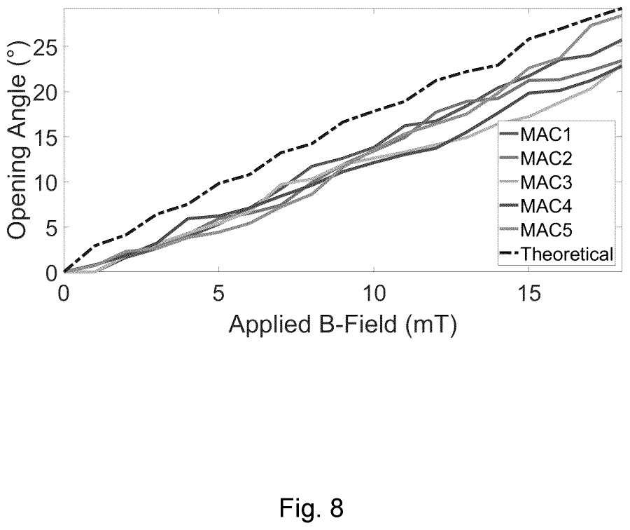

The opening angle was identified from recorded images using the open-source Tracker software [33]. As presented in FIG. 8, the opening angle is a function of the strength of the applied field.

### Example 7: Capsule Chamber Hydrophobicity Analysis

The capturing capability of the capsule during the activation is partially dependent on the hydrophobicity of the chamber site. To study this and assess the potential to improve the surface wettability, the contact angle of water and diiodomethane were measured on five uncoated silicone rubber samples and five coated silicone rubber samples (2 g/L dopamine in Tris-HCl buffer, 24 hours) three times each sample using a contact angle goniometer (OCA 15EC; Dataphysics). The water contact angles (mean±SD, n=15) were 104.5±3.7° and 54.8±6.0° for uncoated and coated silicone rubber, respectively. The surface energies (mean±SD, n=15), calculated as in [34], were 23.4±1.4 and 44.5±4.8 mN/m for uncoated and coated rubber, respectively.

Polydopamine decreased the water contact angle and doubled the surface energy, providing hydrophilic coating of the silicone.

### Example 8: Capsule Tensile Analysis

The silicone rubber by itself is not strong enough to hold the capsule closed during vigorous motion in the stomach of swine model. Therefore, a composite was developed having reinforced carbon fibers aligned longitudinally in the direction of the hinge. A tensile test was performed with two samples each of this composite and silicone rubber using an Instron 4465 universal tensile tester with a crosshead speed of 50 mm/min, based on International Standard ISO-527. The stress-strain curves showed that the maximum tensile strength (12 MPa) of the carbon fiber composite was six times greater than that of the pure rubber (2 MPa).

### Example 9: Acid Resistivity of Capsule

The biological environment of the GI tract is not hostile to silicone rubber, except in the stomach which contains hydrochloric acid with low pH (<4) [35][36]. As the capsule may stay in the stomach for up to several hours during gastric transit, its outer shell should be acid resistant by maintaining its elastic and strength properties. To test the potential for rubber damage in acid, ten dumbbell-shaped samples were prepared according to ASTM 412-416 standard (Die C) for vulcanized rubber and thermoplastic elastomers. Five samples were immersed in hydrochloric acid (pH 1.2) for 24 h. Afterwards, the tensile properties of the 10 samples was measured using an Instron 4465 universal tensile tester with a crosshead speed of 50 mm/min, based on International Standard ISO-527.

Stress-strain curves were analyzed, where a t-test of ultimate tensile strength yielded a p value of 0.22 between samples that were exposed and those unexposed to acid, indicating no significant difference in tensile strength between acid-treated and untreated samples. No significant difference in the tangent modulus was observed and scanning electron microscope images of the rubber surface showed no discernable evidence.

### Example 10: Non-Inhibition of Cell Viability

The compatibility of the capsule for collecting samples and/or deliver agents in a body cavity requires that the capsule materials do not inhibit cell viability of collected samples.

To validate compatibility of capsule materials, the alamarBlue (Thermo Fisher) viability test that is a standard for quantifying cell viability was performed based on the protocol provided in [31]. Capsule material samples were incubated with fibroblast media for 1, 3, and 7 days. Fibroblast cells (3×10⁴ cells/well) were seeded in a 96-well plate and fed with incubated media (500 μL) for 1 day, followed by comparison with unincubated samples. To evaluate cell viability, 50 μL alamarBlue was added to each well. After 4 h incubation at 37° C. in a humidified atmosphere of 5% CO₂ and 95% air, fluorescence was measured with a cyto-fluorometer adapted to a microplate (λₑₓ: 555 nm, λₑₘ: 585 nm) using a SpectraMax i3 multimode microplate reader (Molecular Devices).

Cell viability on the silicone rubber surface of the capsule chamber was tested by exposing cells to polymer for 0 (control), 1, 3, and 7 days. The percentages of cell numbers compared with the control ranged from 98%±0.8% (1 day) to 96%±0.9% (7 days). Thus, the polymer had no measurable effect on cell viability, which may be relevant to its expected effect on sampled microbiota.

### Example 11: Ex Vivo Demonstration of Intestinal Sampling

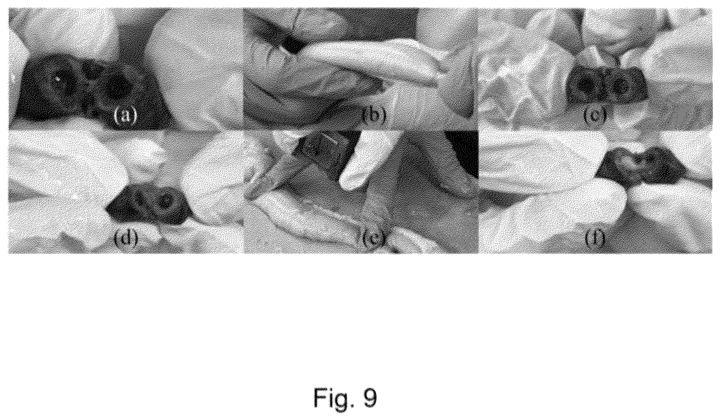

Two meters of proximal jejunum were extracted from a freshly euthanized pig to test capsule sealing and activation. After visual inspection of their chambers to confirm they were empty, five capsules were inserted proximally into the intestine segment by hand, agitated by hand (with motion in all directions), and extracted distally. The sampling chamber contents were then examined subjectively (FIG. 9 (a-c)). The amount of digestive system material that entered into the capsules during this sealing test was insignificant.

An ex-vivo sampling test was also conducted using the intestine segment. Each of the same five capsules was then inserted proximally, moved to the mid-jejunum, and activated using a 2.5 cm cubic external magnet. Then, the capsule was distally removed and the capsule sampled content was observed (FIG. 9 (d-f)). In this sampling test, all five of the chambers contained significant amounts of digestive system material (FIG. 9f).

### Example 12: In Vivo Demonstration of Intestinal Sampling

To test the capability of the capsule to capture samples in a live animal, in vivo experiments were conducted, reviewed and approved by the University of Guelph Animal Care Committee following Canadian Council on Animal Care guidelines [37]. Four capsules were fed with the aid of an injection tube in the pig's mouth to three pigs (age 7-8 weeks, body weight 15-20 kg) with a timing of 0 hr, 4 hr, 6 hr, and 8 hr. Thus 12 capsules in total were fed to the pigs. All capsules in the pigs were activated at the same time after 30.5 h by manual motion of the external magnet around the pig abdomen. The pigs were then euthanized, and the capsules were retrieved, immediately snapped frozen at −40° C., and stored at −90° C. The content of each capsule was then weighed and used for DNA analysis. Eleven of the twelve capsules successfully collected digestive tract material in the range of 18 to 61 mg. The twelfth capsule did not appear to open and collected no digestive tract sample.

### Example 13: Cargo Delivery—In Vitro

The ability of the capsule to blindly deliver cargo within a body cavity was assessed both in vitro and in vivo. For in vitro tests, the capsule was loaded with water and a known quantity of red dye to simulate a drug load. The capsule was placed into a beaker of water, where it was agitated by hand to observe the proper sealing of the capsule to contain the red dye prior to activation. To confirm the activation and drug release, an external permanent magnet was brought close to open the capsule.

To simulate the “blind” activation of the capsule inside the body, the magnet operator was visually shielded from seeing the beaker. The capsule was opened to release the drug. The magnetic field was then removed to close the capsule.

To confirm the quantity of drug released, the concentration of red dye in the beaker was estimated by visually calculating the region of red dye spread and intensity, and compared with a scenario where the same quantity of dye was released directly into a similar beaker of water.

A second control experiment was run where a dye-loaded capsule was placed into the beaker, agitated, but not opened and subsequently removed from the beaker. This dye concentration measured from this second control test confirms that the capsule can deliver the drug only to the target location and does not leak prior to activation.

A third experiment was run where a dye-loaded capsule was placed into the beaker, and opened slowly under a weaker magnetic field of only 5 mT strength. The concentration of red dye in the beaker was estimated by visually calculating the region of red dye spread and intensity.

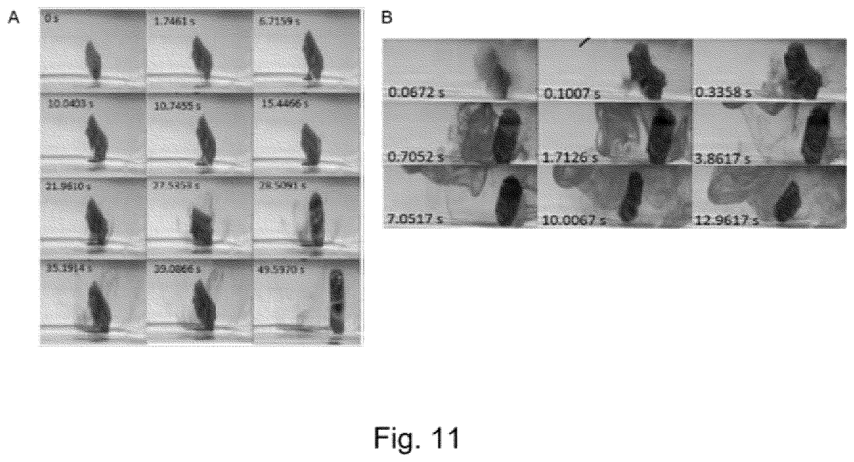

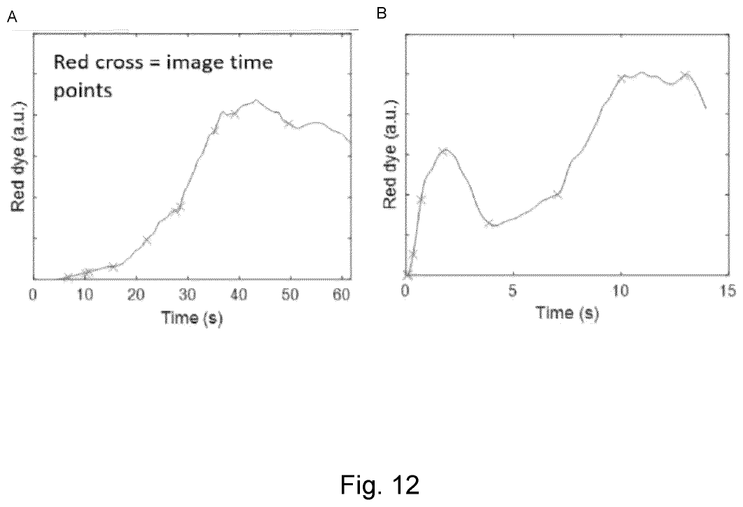

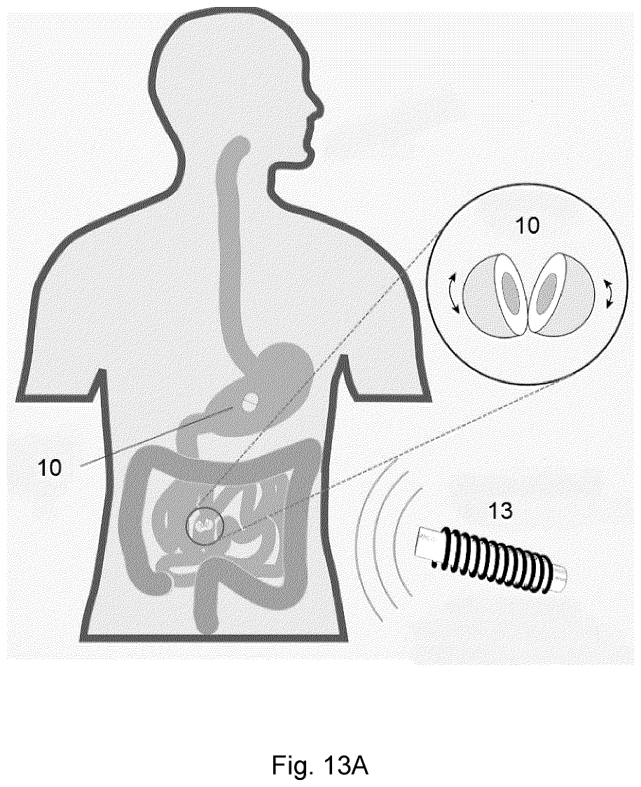

The results are shown in FIGS. 11 and 12. FIGS. 11A and 12A demonstrate the slow release of the drug payload when blindly activated by the external magnetic field, and FIGS. 11B and 12B demonstrate the immediate quick release of the drug payload.

### Example 14: Cargo Delivery—In Vivo

A live animal model test was performed to confirm that the capsule can release a simulated drug payload inside the intestine of a live pig. The capsule was pre-loaded with a blue dye and then orally administered to the pig. After waiting for the capsule to move partially through the GI tract by waiting 4 hours, the capsule was opened wirelessly by application of a magnetic field. The magnetic field was then removed, closing the capsule. The pig was slaughtered and dissected. The presence of dye in the target location of the GI tract was confirmed visually. A control test was run with a dye-loaded capsule that was not activated. After the pig was euthanized and dissected, the lack of released dye in the intestine was confirmed visually. The results of this in vivo test demonstrated the ability of the capsule to be blindly activated to deliver a cargo to a target location.

The disclosures of all patents, patent applications, publications and database entries referenced in this specification are hereby specifically incorporated by reference in their entirety to the same extent as if each such individual patent, patent application, publication and database entry were specifically and individually indicated to be incorporated by reference.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1. A magnetically actuated capsule comprising two portions pivotally connected to each other for allowing reconfiguration of the capsule between a closed configuration and an open configuration, the portions defining a chamber enclosed within the capsule when the capsule is in the closed configuration, and defining an aperture exposing the chamber to the exterior of the capsule when the capsule is in the open configuration, each portion comprising at least one permanent magnet having a magnetic moment disposed in a direction opposite to the magnetic moment of the at least one permanent magnet in the other portion such that:
(i) an inter-magnet attraction force is generated to bias the capsule to the closed configuration, and
(ii) the capsule in the closed configuration has a net magnetic moment that aligns with an externally applied magnetic field to actuate reconfiguration of the capsule to the open configuration when said magnetic field is applied.

2. The capsule of claim 1, wherein the capsule further comprises a seal for sealing the chamber when the capsule is in the closed configuration, the seal being disposed between the portions of the capsule in the closed configuration.

3. The capsule of claim 1, wherein the capsule in the closed configuration comprises a cylindrical midsection, wherein the portions are pivotally connected at a pivot point on the cylindrical midsection of the capsule, and wherein in the closed configuration the magnetic moment of the at least one permanent magnet in each portion is about parallel to a line which intersects the pivot point and which about perpendicularly intersects the cylinder axis of the cylindrical midsection of the capsule.

4. The capsule of claim 3, wherein the at least one permanent magnet in each portion is disposed within the portion at an angle αₒ relative to the line which intersects the pivot point, such that the net magnetic moment of the capsule self-aligns with the externally applied magnetic field when said magnetic field is applied.

5. (canceled)

6. The capsule of claim 1, wherein the portions are pivotally connected by a hinge.

7. The capsule of claim 1, wherein the capsule comprises a soft outer shell.

8. (canceled)

9. The capsule of claim 1, wherein the capsule is configured to be swallowed and passed through the intestinal tract of a subject.

10. (canceled)

11. (canceled)

12. The capsule of claim 9, wherein said capsule is adapted for collecting a sample from the intestinal tract or delivering one or more agents to the intestinal tract.

13. (canceled)

14. (canceled)

15. A system comprising the capsule of claim 1 and an external magnet for generating a magnetic field, wherein application of the magnetic field remotely actuates alignment of the capsule with the externally applied magnetic field to activate reconfiguration of the capsule from the closed configuration to the open configuration.

16. A method of collecting a sample from the intestinal tract of a subject, the method comprising:
(a) providing a capsule in the closed configuration of claim 1 for ingestion by the subject;
(b) applying an external magnetic field to the subject to actuate reconfiguration of the capsule to the open configuration, thereby exposing the chamber of the capsule to the exterior of the capsule for collection of the sample in the chamber;
(c) removing the external magnetic field to actuate reconfiguration of the capsule to the closed configuration, thereby enclosing the sample in the chamber; and
(d) allowing the capsule to transit through the intestinal tract for recovery by stool passage.

17. The method of claim 16, further comprising the step of
(e) analyzing the sample contained in the recovered capsule so as to diagnose the disease or condition in the subject.

18. A method for delivering an agent to a subject, the method comprising:
(a) providing a capsule in the closed configuration of claim 1, the chamber of the capsule containing the agent;
(b) administering the capsule to the subject by ingestion;
(c) allowing the capsule to transit through the subject's intestinal tract; and
(d) applying an external magnetic field to the subject to actuate reconfiguration of the capsule to the open configuration to dispense the agent contained in the chamber into the intestinal tract of the subject.

19. (canceled)

20. The method of claim 18, wherein the agent is a biological agent or a non-biological agent.

21. (canceled)

22. (canceled)

23. The method of claim 16, wherein step (a) comprises providing more than one capsule to the subject to ingest in intervals.

24. A method of manufacturing the magnetically actuated capsule of claim 1, the method comprising:
(a) for each portion, providing a mold for forming the portion, the mold comprising a chamber ridge for forming a capsule chamber;
(b) mounting at least one permanent magnet to the mold, the at least one permanent magnet being positioned in the mold such that the two portions are biased to magnetically connect to each other to form the capsule;
(c) pouring a polymer in a liquid state into the mold and allowing the polymer to solidify, thereby forming the portion comprising the at least one permanent magnet; and
(d) contacting the two portions to each other so as to allow the portions to magnetically connect to each other to form the capsule.

25. The method of claim 24, wherein the mold further comprises a magnet holder for mounting the at least one permanent magnet, and wherein in step (b) the at least one permanent magnet is mounted on a magnet holder.

26. The method of claim 24, wherein the method further comprises pivotally joining the portions to each other with a hinge at a pivot point.

27. The method of claim 24, wherein said pivotally joining the portions to each other with a hinge at a pivot point comprises pouring into a hinge mold a composite in a liquid state, the composite being a reinforced form of the polymer used to create the portions of the capsule and being flexible in the solid state, placing the capsule in the hinge mold containing the composite such that a longitudinal strip of the capsule surface spanning both portions contacts the composite, and allowing the composite to solidify, thereby pivotally connecting the portions of the capsule to each other with a flexible composite hinge at the pivot point.
